(19) United States
(12) Patent Application Publication
(10) Pub. No.: US 2006/0134775 A1
(43) Pub. Date: Jun. 22, 2006

(54) Systems, illumination subsystems, and methods for increasing fluorescence emitted by a fluorophore

(76) Inventor: Jesse Phillips, Round Rock, TX (US)

Correspondence Address: DAFFER MCDANEIL LLP, P.O. BOX 684908, AUSTIN, TX, 78768, US

(21) Appl. No.: 11/305,805

(22) Filed: Dec. 16, 2005

Related U.S. Application Data
(60) Provisional application No. 60/637,355, filed on Dec. 17, 2004.

(51) Int. Cl. C12M 1/34; G01N 33/551

(52) U.S. Cl. 435/287.2; 436/524

(57) ABSTRACT

Systems, illumination subsystems, and methods for increasing fluorescence emitted by a fluorophore are provided. One system configured to measure fluorescence of particles includes an illumination subsystem configured to illuminate the particles with light having linear polarization oriented in a non-vertical direction, circular polarization, or elliptical polarization. The polarization of the light causes fluorescence emitted by the fluorophore to be brighter than fluorescence emitted by the fluorophore when illuminated with linearly polarized light oriented in a predominantly vertical direction or non-polarized light. The system also includes a detection subsystem configured to generate output signals responsive to the fluorescence emitted by the fluorophore.

### PRIORITY CLAIM

This application claims priority to U.S. Provisional Application No. 60/637,355 entitled “Methods and Systems for Increasing Fluorescence from a Fluorophore,” filed Dec. 17, 2004, which is incorporated by reference as if fully set forth herein.

### BACKGROUND OF THE INVENTION

1. Field of the Invention The present invention generally relates to systems, illumination subsystems, and methods for increasing fluorescence emitted by a fluorophore. Certain embodiments relate to altering the polarization of the light used to illuminate particles to which a fluorophore is attached or in which a fluorophore is incorporated thereby increasing fluorescence emitted by the fluorophore.

2. Description of the Related Art

The following description and examples are not admitted to be prior art by virtue of their inclusion in this section.

Optical systems have been and will increasingly be used to obtain measurements for a number of different samples such as biological samples. One particular optical-based system that is becoming increasingly important in the biological assay field is the flow cytometer, which allows scientists to examine a sample for a relatively large number of analytes in a relatively short amount of time. As with all optical systems, the accuracy of the measurements of a flow cytometer is dependent in large part on the signal-to-noise ratio (SNR) that can be obtained using the flow cytometer. In particular, as the SNR increases, the measurement accuracy increases.

The optical design and configuration of flow cytometers, like all optical systems, have, therefore, been developed taking into consideration the SNR requirements. For instance, one way that the SNR can be increased is to increase the brightness of the light source used to illuminate the particles being measured. In particular, as the amount of light available for measurements increases, the SNR of the measurements will generally increase. In this manner, high intensity light sources such as lasers are often used in flow cytometers. Other ways to increase the SNR of a flow cytometer may include selection and configuration of focusing optics, collecting optics (for collecting light scattered or emitted from the sample), and detectors included in the flow cytometer. Although improvements in the SNR of flow cytometers have been made since flow cytometers were first introduced, the search for the maximum SNR possible is an ongoing task.

Currently, linearly polarized light is used to illuminate flow cells in flow cytometers for the purposes of scatter and fluorescence measurements. For example, U.S. Pat. No. 5,017,497 to de Grooth et al., which is incorporated by reference as if fully set forth herein, describes using linearly polarized light at a vertical angle in the plane that is orthogonal to the beam axis. However, de Grooth et al. provide no definition of the polarization ratio for “linearly polarized” light. In addition, altering the state of the polarization (such as to elliptical) of the illumination light for purposes of fluorescence measurements has not been previously performed.

Optimizing the samples that are measured in flow cytometers such as fluorescently dyed beads and fluorescently dyed reagents has also been explored as a means for increasing the SNR of flow cytometer measurements. However, the materials that are available for use in flow cytometer measurements are often somewhat limited. For instance, the materials must be compatible with the sample that will be examined. In other words, preferably, the materials of the beads and fluorescent dyes do not alter the sample being measured or vice versa. In addition, the materials are preferably compatible with the design of the flow cytometer. For instance, preferably, the fluorescent dyes are excited at the wavelength(s) of at least one light source of the flow cytometer. In addition, the material of the beads preferably is not altered by the wavelength(s) of light they will be exposed to by the flow cytometer. For at least these reasons, it may be more attractive and less complicated to try to increase the SNR of flow cytometer measurements by altering the optical design and configuration of the flow cytometer as opposed to altering the materials that will be used in the samples.

Accordingly, it would be advantageous to develop systems, illumination subsystems, and methods for increasing fluorescence emitted by a fluorophore by altering one or more parameters of a flow cytometer or other fluorescence based imaging and/or measurement system.

### SUMMARY OF THE INVENTION

The following description of various system, illumination subsystem, and method embodiments is not to be construed in any way as limiting the subject matter of the appended claims.

An embodiment relates to a system configured to measure fluorescence of particles. The system includes an illumination subsystem configured to illuminate the particles with light having linear polarization oriented in a non-vertical direction, circular polarization, or elliptical polarization. A fluorophore is attached to or incorporated into the particles. The polarization of the light causes fluorescence emitted by the fluorophore to be brighter than fluorescence emitted by the fluorophore when illuminated with linearly polarized light (e.g., from either a laser or non-laser light source) oriented in a predominantly vertical direction or non-polarized light (e.g., from any light source). The system also includes a detection subsystem configured to generate output signals responsive to the fluorescence emitted by the fluorophore.

In one embodiment, the illumination subsystem includes one or more lasers. In another embodiment, the illumination subsystem includes one or more non-laser light sources selected from the group consisting of light emitting diodes (LEDs), arc lamps, fiber illuminators, and light bulbs.

In one embodiment, the polarization has a polarization ratio (i.e., the ratio of the semimajor axis of the polarization to the semiminor axis of the polarization) of less than 100:1 with any orientation. In another embodiment, the polarization has a polarization ratio of greater than 100:1 with a non-vertical orientation. In contrast, linear polarization oriented in a predominantly vertical direction has a polarization ratio of greater than 100:1 and is oriented predominantly in the vertical direction. Linear polarization is defined herein as a polarization state having a polarization ratio of greater than 100:1, regardless of the orientation of the semimajor axis with respect to the vertical direction.

Non-linear polarization that is elliptically polarized and oriented in any direction with respect to vertical has a polarization ratio of less than 100:1; otherwise, the elliptical polarization would be considered linear polarization with some orientation. Technically, all polarization could be referred to as elliptical. If polarization is defined in this manner, circular polarization would then be a special case of elliptical polarization in which both the semimajor and semiminor axes have the same magnitude. Linear polarization would then also be a special case of elliptical polarization in which the magnitude of the semimajor and semiminor axes has a ratio of greater than 100:1.

In one embodiment, the fluorophore includes R-phycoerytherin (R-PE). In another embodiment, the fluorophore includes an organic or non-organic dye. In some embodiments, the particles are configured to emit fluorescence. In one such embodiment, the polarization of the light causes the fluorescence emitted by the particles to be brighter than the fluorescence emitted by the particles when illuminated with the linearly polarized light oriented in the predominantly vertical direction or non-polarized light.

In one embodiment, the system is configured as a flow cytometer. In a different embodiment, the system is configured as a fluorescence imaging system. Each of the embodiments of the system described above may be further configured as described herein.

Another embodiment relates to an illumination subsystem configured to provide illumination for a measurement system. The illumination subsystem includes a light source configured to generate light. The illumination subsystem also includes a polarization component configured to alter the polarization of the light before the light illuminates particles during measurements performed by the measurement system. The altered polarization is linear polarization oriented in a non-vertical direction, circular polarization, or elliptical polarization. A fluorophore is attached to or incorporated into the particles. The altered polarization causes fluorescence emitted by the fluorophore to be brighter than fluorescence emitted by the fluorophore when illuminated with linearly polarized light oriented in a predominantly vertical direction or non-polarized light.

In one embodiment, the light source includes one or more lasers. In another embodiment, the light source includes one or more non-laser light sources selected from the group consisting of LEDs, arc lamps, fiber illuminators, and light bulbs. In some embodiments, the polarization component includes a half-wave retarder, a quarter-wave retarder, a retarder stack, or some combination thereof.

In one embodiment, the altered polarization has a polarization ratio of less than 100:1 with any orientation. In a different embodiment, the altered polarization has a polarization ratio of greater than 100:1 with a non-vertical orientation. In another embodiment, the fluorophore includes R-PE. In a different embodiment, the fluorophore includes an organic or non-organic dye.

In one embodiment, the measurement system is configured as a flow cytometer. In a different embodiment, the measurement system is configured as a fluorescence imaging system. Each of the embodiments of the illumination subsystem described above may be further configured as described herein.

An additional embodiment relates to a method for increasing fluorescence emitted by a fluorophore attached to or incorporated into particles. The method includes altering the polarization of light before the light illuminates the particles during a measurement. The altered polarization is linear polarization oriented in a non-vertical direction, circular polarization, or elliptical polarization. In one embodiment, the altered polarization has a polarization ratio of less than 100:1 with any orientation. In a different embodiment, the altered polarization has a polarization ratio of greater than 100:1 with a non-vertical orientation. Each of the embodiments of the method described above may include any other step(s) described herein.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a schematic diagram illustrating a perspective view of one example of an optical configuration for a flow cytometer;

FIG. 2 is a schematic diagram illustrating a perspective view of an embodiment of an optical configuration for a flow cytometer;

FIGS. 3-6 are graphs illustrating data that was obtained by illuminating particles with light, both without altering the polarization of the light and with altering the polarization of the light; and FIGS. 7-8 are schematic diagrams illustrating a cross-sectional view of various embodiments of a system configured to measure fluorescence of particles.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

The following description generally relates to systems, illumination subsystems, and methods for obtaining increased fluorescence from fluorophores such as R-phycoerytherin (R-PE) by altering the polarization state and/or orientation (e.g., the degree of elliptical polarization) of a light beam used to illuminate the fluorophores.

Although embodiments are described herein with respect to particles, it is to be understood that the systems, illumination subsystems, and methods may also be used for microspheres, polystyrene beads, microparticles, gold nanoparticles, quantum dots, nanodots, nanoparticles, nanoshells, beads, microbeads, latex particles, latex beads, fluorescent beads, fluorescent particles, colored particles, colored beads, tissue, cells, micro-organisms, organic matter, or any non-organic matter. The particles may serve as vehicles for molecular reactions. Examples of appropriate microspheres, beads, and particles are illustrated in U.S. Pat. No. 5,736,330 to Fulton, U.S. Pat. No. 5,981,180 to Chandler et al., U.S. Pat. No. 6,057,107 to Fulton, U.S. Pat. No. 6,268,222 to Chandler et al., U.S. Pat. No. 6,449,562 to Chandler et al., U.S. Pat. No. 6,514,295 to Chandler et al., U.S. Pat. No. 6,524,793 to Chandler et al., and U.S. Pat. No. 6,528,165 to Chandler, which are incorporated by reference as if fully set forth herein. The systems, illumination subsystems, and methods described herein may be used with any of the microspheres, beads, and particles described in these patents. In addition, microspheres for use in flow cytometry may be obtained from manufacturers such as Luminex Corporation, Austin, Tex. The terms “particles,” “beads,” and “microspheres” are used interchangeably herein.

Although some embodiments are described herein with respect to lasers, it is to be understood that the system, illumination subsystem, and method embodiments may also be used with one or more lasers and/or one or more non-laser light sources. These non-laser light sources include light emitting diodes (LEDs), arc lamps, fiber illuminators, light bulbs, and any other suitable non-laser light sources known in the art. It is to be understood that light generated by any coherent, partially coherent, or non-coherent light source can be conditioned or altered to have a selected polarization state and/or orientation by appropriate optical filter and/or polarization components, which may be configured as described herein.

Turning now to the drawings, it is noted that the figures are not drawn to scale. In particular, the scale of some of the elements of the figures is greatly exaggerated to emphasize characteristics of the elements. It is also noted that the figures are not drawn to the same scale. Elements shown in more than one figure that may be similarly configured have been indicated using the same reference numerals.

Flow cytometers typically utilize a linearly polarized laser beam to illuminate a sample that is contained in a flow cell. Light generated by most lasers utilized in flow cytometers is linearly polarized with a minimum polarization ratio of 100:1. Linearly polarized light generated by actual lasers varies significantly in polarization ratio from 100:1 to over 800:1. The polarization ratio describes the ratio of the magnitude of the axis components of elliptical polarization. A light wave can be modeled mathematically as two orthogonal wave components. Depending upon the relative phase of these two orthogonal waves, the resulting instantaneous polarization state vector will “trace” an ellipse as the lightwave travels a wavelength. The polarization ratio is the ratio of the magnitude of the semimajor axis component to the magnitude of the semiminor axis component (orthogonal to the semimajor axis component) of this elliptical polarization. Therefore, the term “linearly polarized” is not an absolute term, but is relative, and refers to a linearly polarized light beam with a polarization ratio that exceeds a minimum of 100:1, but which may vary significantly in actual practice. The orientation of the semimajor axis will define the orientation of the polarization.

Experimental data has demonstrated that applying a certain amount of phase retardation to a laser beam significantly increases the amount of fluorescence emitted by certain fluorophores when illuminated with the laser beam. Phase retardation that can increase the fluorescence emitted by the fluorophores alters the polarization state of the laser beam to an altered polarization state. This altered polarization state can include linear polarization with an angular orientation with respect to vertical, circular polarization, or elliptical polarization. The orientation of the elliptical polarization may have a significant component away from the vertical direction.

The term “vertical” or “vertical direction” can be defined differently for different measurement systems. For instance, in flow cytometers, polarization oriented predominantly in the vertical direction is generally defined herein as polarization having a semimajor axis that is substantially parallel to the flow of particles through the flow cytometer at the point at which the particles are illuminated. In this manner, a laser can be arranged in any position and orientation, and through the use of mirrors, etc., the beam can be routed, reflected and directed such that when the beam impinges upon the particles, the polarization of the beam has a selected orientation. In this situation, the orientation of the polarization may vary along the illumination path as long as the polarization state of the light that impinges on the particles has a selected orientation.

In a fluorescence imaging system (or “plate reader”), the particles may have substantially stationary positions on a substrate. Therefore, in such systems, the vertical direction cannot be defined with respect to the flow of the particles. For some imaging measurement systems such as those described further herein, light may be directed at a substantially normal angle of incidence to a plane in which particles are positioned. In such systems, at the point at which the particles are illuminated, the polarization vector of the light will be substantially parallel to this plane regardless of orientation. In this manner, the orientation of the polarization can be defined with reference to a different axis or plane of the measurement system. In this configuration, the orientation of the polarization of the incident light will give rise to a “dominant” polarization component (or orientation) of the fluoresced light by certain fluorescent dyes. Dyes such as the calibrator dye used in Luminex calibration beads are small and linear. Such dyes reproduce the polarization of the incident light to a significant and measurable degree in their fluoresced light. If this fluoresced light were to be split using a polarization beamsplitter, linearly polarized fluorescent light aligned with the polarization of the incident light will either pass through or be reflected by the polarization beamsplitter depending upon the orientation of the beamsplitter. If this polarization beamsplitter were oriented such that the beam-splitting surface is parallel to the linear polarization of the fluorescent light, then this would be the defining geometry for linearly polarized incident light for the plate reader configuration with incident light at normal incidence. In any case, the polarization of the light emitted by the light source may be linear (oriented in any direction) and may be modified either by altering the orientation of the polarization or by altering the state of the polarization to circular or elliptical (oriented in any direction) according to embodiments described herein.

A fluorescence imaging measurement system may alternatively be configured such that light is directed to the particles at an angle of incidence that is oblique or non-normal to the plane of the plate which holds the sample particles. In this configuration, the orientation of the polarization of the incident light can be defined as vertical when the semimajor axis of the polarization is substantially parallel to the plane of incidence. The plane of incidence is that plane that contains the incident ray, the reflected ray and any ray that is partially or wholly transmitted through or into the medium beyond the optical surface (or would be transmitted if the surface medium is non-transmissive). This polarization will then be predominantly “p” polarization.

According to one embodiment, therefore, a method for increasing fluorescence emitted by a fluorophore attached to or incorporated into particles includes altering the polarization of light before the light illuminates the particles during a measurement. The altered polarization is linear polarization oriented in a non-vertical direction, circular polarization, or elliptical polarization. In one embodiment, the altered polarization has a polarization ratio of less than 100:1 with any orientation. In another embodiment, the altered polarization has a polarization ratio of greater than 100:1 with a non-vertical orientation. In contrast, linear polarization oriented in a predominantly vertical direction has a polarization ratio of greater than 100:1 and is oriented in the vertical direction. Linear polarization oriented in a non-vertical direction (i.e., linear polarization having its semimajor axis oriented in a non-vertical direction) has.a polarization ratio of greater than 100:1 but the ratio of vertical to horizontal polarization is less than 100:1. Regardless of the orientation of the linear polarization, the polarization ratio is greater than 100:1. That is, given the predominant orientation of linear polarization, the ratio of the semimajor axis polarization component to the polarization component orthogonal to the semimajor axis is greater than 100:1.

Non-linear polarization that is elliptically polarized and oriented in any direction has a polarization ratio of less than 100:1; otherwise, the elliptical polarization would be considered linear polarization with some orientation. Technically, all polarization could be referred to as elliptical. If polarization is defined in this manner, circular polarization would then be a special case of elliptical polarization in which both the semimajor and semiminor axes have the same magnitude. Linear polarization would then also be a special case of elliptical polarization in which the semimajor and semiminor axes have a ratio of greater than 100:1.

In some embodiments, the fluorophore includes R-PE. In other embodiments, the fluorophore includes an organic or non-organic dye. The organic or non-organic dye may include any appropriate dye known in the art, some examples of which are included in the patents incorporated by reference above. The fluorophore may be attached to or incorporated into the particles in any manner known in the art, some examples of which are included in the patents incorporated by reference above. The fluorophore may be used for classification or identification of the particles (e.g., identification of a subset of a population to which the particles belong). Alternatively, the fluorophore may be used for identification, determination, or quantification of a reaction that has taken place on the surface of the particles.

Each of the embodiments of the method described above may include any other step(s) described herein. In addition, each of the embodiments of the method described above may be performed by any of the illumination subsystems or systems described herein. Furthermore, each of the embodiments of the method described above has all of the advantages of other embodiments described herein.

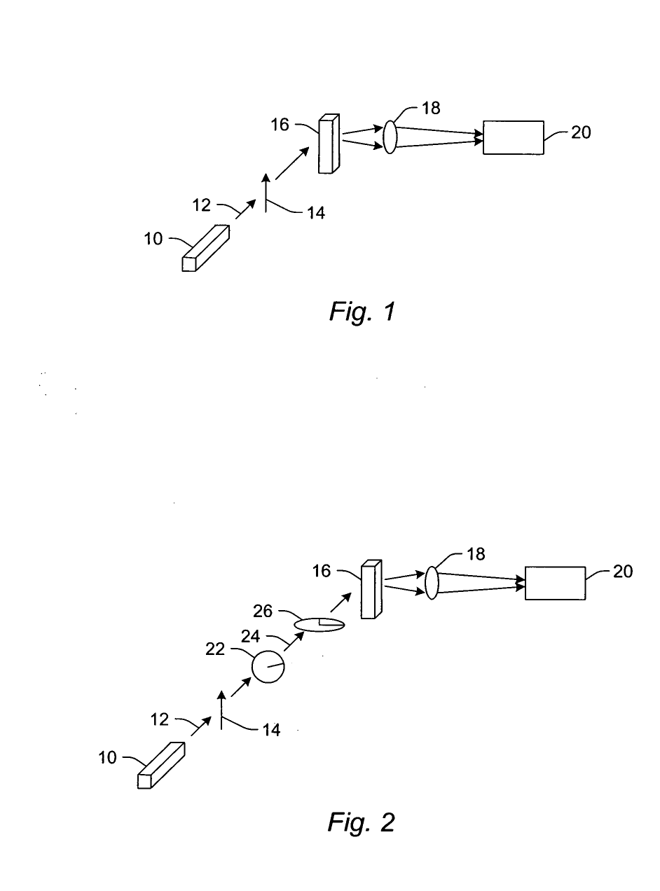

The technique described above can be applied to existing flow cytometers that are configured to illuminate particles using lasers that generate beams of predominantly linearly polarized light. One optical configuration used in existing flow cytometers is shown in FIG. 1. As shown in FIG. 1, the optical configuration includes light source 10, which may be a laser or any other suitable light source known in the art. The light source is configured to generate linearly polarized light oriented in a predominantly vertical direction. In particular, light 12 generated by light source 10 has polarization 14, which is linear and oriented in a predominantly vertical direction (i.e., a direction substantially parallel to the flow of particles (not shown) through cuvette 16 ).

Light 12 is directed to cuvette 16 such that the light illuminates particles flowing through the cuvette. Light 12 may also be focused to cuvette 16 by one or more optical components (not shown) such as a focusing lens disposed between light source 10 and cuvette 16. Light emitted and/or scattered by the particles, materials attached thereto, or materials incorporated therein is collected by collection optics 18. Collection optics 18 is configured to direct the collected light to detector 20. Detector 20 generates output signals responsive to the emitted and/or scattered light collected by collection optics 18. Cuvette 16, collection optics 18, and detector 20 may include any appropriate components known in the art.

In the embodiment of the flow cytometer optical configuration shown in FIG. 2, the optical configuration shown in FIG. 1 is modified such that the polarization state of the illumination is modified by phase retardation to, for example, an elliptical or circular polarized state to increase, and possibly even maximize, the fluorescence emitted by a fluorophore such as R-PE. Elements shown in FIG. 2 that may be configured as described above with respect to FIG. 1 are not described further herein for the sake of brevity.

The optical configuration embodiment shown in FIG. 2 includes an illumination subsystem configured to provide illumination for a measurement system. In particular, like the optical configuration shown in FIG. 1, the optical configuration shown in FIG. 2 includes light source 10. Light source 10 is configured to generate light 12 as described above. In one embodiment, light source 10 is a laser. The laser may be any suitable laser known in the art. In addition, the optical configuration may include more than one laser (not shown). In other embodiments, light source 10 is a non-laser light source selected from the group consisting of a LED, an arc lamp, a fiber illuminator, and a light bulb. The light source may include any such appropriate light sources known in the art. In addition, the optical configuration shown in FIG. 2 may include more than one non-laser light source (not shown). Furthermore, the optical configuration may include one or more lasers and one or more non-laser light sources. The light source may be further configured as described herein. Unlike the optical configuration shown in FIG. 1, the optical configuration shown in FIG. 2 includes polarization component 22 disposed between light source 10 and cuvette 16 . Polarization component 22 is configured to alter the polarization of light 12 before the light illuminates particles (not shown in FIG. 2) during measurements performed by the measurement system. In other words, polarization component 22 is disposed in the optical path of light 12 to change the polarization state and/or orientation of the light. For example, as shown in FIG. 2, light 24 exiting polarization component 22 has altered polarization 26, which is shown as elliptical polarization oriented in a direction that is different than the direction in which polarization 14 is oriented. However, altered polarization 26 may be linear polarization oriented in a non-vertical direction, circular polarization, or elliptical polarization (oriented in any direction with respect to vertical). The altered polarization may have a polarization ratio of less than 100:1 with any orientation. The altered polarization may alternatively have a polarization ratio of greater than 100:1 with a non-vertical orientation. Since most lasers generate light having varying linear polarization ratios (when they are configured to generate linearly polarized light), the specific amount of phase retardation applied to light 12 by polarization component 22 can vary from laser to laser and can be selected during system alignment.

In embodiments described herein, therefore, a polarization component such as a wave plate, polarization retarder, or other polarization component or combination of polarization components described herein is placed between the light source (e.g., a laser) and the illumination target (e.g., the particles being measured in the case of a flow cytometer) to alter the polarization of a light beam (e.g., a linearly polarized beam). In some embodiments, the polarization component includes a half-wave retarder, a quarter-wave retarder, a polarization component configured to have any combination of phase retardance functions such as a retarder stack, any other optical component known in the art that can be used to alter the polarization of the light generated by light source 10, or some combination thereof. This retarder can be placed in the illumination path before or after focusing optics (not shown), if focusing optics are included in the illumination subsystem. In addition, although system and illumination subsystem embodiments are described herein as including a polarization component, it is to be understood that the systems and illumination subsystems may include more than one polarization component (not shown) positioned in the illumination path such that the polarization components in series alter the polarization of a light beam generated by a light source.

A fluorophore (not shown) is attached to or incorporated into the particles that flow through cuvette 16 . The fluorophore may include R-PE. Alternatively, the fluorophore may include an organic or non-organic dye. The organic or non-organic dye may include any appropriate dye known in the art. The fluorophore may be further configured as described herein. The altered polarization preferably causes fluorescence emitted by the fluorophore to be brighter than fluorescence emitted by the fluorophore when illuminated with linearly polarized light (e.g., from either a laser or non-laser light source) oriented in the predominantly vertical direction or non-polarized light (e.g., from any light source). In other words, polarization component 22 alters the polarization ratio and/or orientation (or the relative phase of the polarization) of light 12 to a selected polarization state and/or orientation, which increases the measured fluorescence emission of the target fluorophore.

In an additional embodiment, the particles themselves are configured to emit fluorescence. In one such embodiment, the polarization of the light exiting polarization component 22 that illuminates the particles causes the fluorescence emitted by the particles to be brighter than the fluorescence emitted by the particles when illuminated with linearly polarized light oriented in the predominantly vertical direction or non-polarized light. As described above, altering the relative phase of the polarization of light 12 may result in an altered polarization state and/or orientation such as linear polarization with a non-vertical orientation, circular polarization, or elliptical polarization oriented at any angle with respect to vertical.

In one embodiment, the optical configuration shown in FIG. 2 is used to provide illumination for a measurement system that is configured as a flow cytometer. The optical configuration shown in FIG. 2 has a number of advantages over other optical configurations currently used in flow cytometers. For example, to increase the fluorescence emitted by a fluorophore, only a polarization component such as an polarization waveplate is inserted into the illumination path of the optical configuration. In this manner, the optical configuration of currently used flow cytometers can be altered in a substantially simple manner to effectuate significant increases in the signal-to-noise ratio and therefore the accuracy of the flow cytometers. In addition, in some embodiments, the optical configuration shown in FIG. 2 may be configured to provide illumination for a measurement system that is configured as a fluorescence imaging system such as the imaging system described further herein. Such embodiments will also have the advantages of the optical configuration shown in FIG. 2. Furthermore, the optical configuration shown in FIG. 2 may be used in any measurement system that performs measurements of fluorescence emitted by a fluorophore and that will benefit from higher signal-to-noise ratios provided by the embodiments described herein. The optical configuration shown in FIG. 2 may be further configured as described herein.

The exact amount of phase retardance applied to the polarization of the illumination that results in the optimal fluorophore absorption/emission may vary depending upon, for example, flow cytometer characteristics (e.g., the wavelength and other characteristics of the light source) and characteristics of the fluorophore. Results are presented below (see, for example, Table 1) that document an increase in measured fluorescence emission by R-PE using illumination at 532 nm in the Luminex LX100 flow cytometer, which is commercially available from Luminex Corporation.

It is to be understood that the systems, illumination subsystems, and methods described herein are not limited to a particular mechanism by which the increased fluorescence is achieved (i.e., the increase in fluorescence signal response may be caused by increased absorption mechanisms and/or may be partially caused by more efficient emission and/or emission detection). However, several possible mechanisms are described below.

The fluorescing reporter dye utilized in the LX100 flow cytometer is R-PE. R-PE molecules are relatively large and have a molecular weight of 240,000 Daltons. Since these molecules are attached to particles via other molecules (e.g., via reaction products), each R-PE molecule can be considered to have its “center” spaced from the surface of the particle. At least one outer edge of the R-PE molecule will be a considerable distance beyond its center away from the particle.

When such a particle is contained in a moving column of liquid such as saline, the particle is carried, or “pushed,” in the direction of the liquid movement by hundreds to thousands of liquid molecules, each of which is under pressure. This pressure is responsible for the movement of the liquid. In addition, the constant application of this pressure may cause a majority (or at least some) of the R-PE molecules attached to the particle to be oriented in a direction that tends toward the direction of the fluid flow (i.e., the molecules are “bent” in the upward direction). This orientation, when summed for all R-PE molecules located across the entire surface of a microsphere, results in a net orientation vector. This orientation vector may affect which polarization state and/or orientation is optimal for the illumination.

Standard Luminex calibration and control particles are coupled to or include a calibration dye that is a member of the squaraine class of dyes. This calibration dye is not affected by the polarization state of the illuminating beam in the same way as R-PE. This non-responsiveness is most likely due to the fact that the calibration dye is a synthetic dye, which is relatively small and “flat” (or linear) in form factor and is distributed randomly throughout the interior of the particles. Thus, there is no net orientation of the calibration dye molecules across the particles. Therefore, if the absorbing dipole moments of the calibration dye molecules are integrated together, the net result will be approximately zero. In addition, since the calibration dye is a small, linear molecule, the calibration dye may tend to respond more efficiently to linearly polarized light than to light having other polarization states.

Other mechanisms may also contribute to the observed increase in fluorescence emitted by R-PE when illuminated with light other than linearly polarized light oriented in a predominantly vertical direction or non-polarized light. For example, R-PE, like all proteins, has a helical or chiral structure. This helical or chiral structure may be partially responsible for the change in responsiveness (i.e., brightness of measured fluorescence emission) of R-PE due to a corresponding change in the degree of elliptical polarization of the illumination. Proteins such as R-PE are composed of amino acids, which are often optically active substances in that they are “rotatory” in the sense of their effect on incoming light and the polarization state of resultant scattered light. The combination of the absorbing chromophores, their net dipole moment, and their chiral structure may be evaluated to determine the polarization state and orientation of illumination that will result in the most efficient absorption of the light by the fluorophore.

Another potential mechanism for the fluorescence increase described herein is the Forster Resonance Energy Transfer (FRET) that takes place in R-PE. R-PE has multiple absorbing chromophores, resulting in a broad absorption spectrum. However R-PE has a relatively narrow emission spectrum, indicating a single emitting chromophore. The emitting chromophore obtains energy from the absorbing chromophores through FRET. This process takes place until the fluorescing chromophore emits photons. FRET between adjacent chromophores occurs when the oscillating dipole moments of the chromophores are in relatively close proximity to each other and energy transfer can occur via the electric fields of the dipole moments (i.e., no photons are emitted during the FRET process). In R-PE, the absorbing chromophore does not emit a photon, but transfers the absorbed energy to a neighboring chromophore. The change in the polarization state and/or orientation of the illumination may not only affect the initial absorbing chromophore but may also increase the efficiency of the FRET process possibly by aligning dipole moments between adjacent chromophores.

Additionally, light that is circularly polarized (or elliptically polarized), is imbued with a net angular momentum whereas linearly polarized light has no net angular momentum. Circularly polarized light will cause the electrons in the absorbing material to have a partial circular (or elliptical) motion in response to the force generated by the electrical field. This motion imparts a net angular momentum to the excited electrons whereas linearly polarized light imparts no angular momentum to the excited electrons. The angular momentum imparted to the electrons depends upon the handedness (right or left) of the circular (or elliptical) polarization relative to the direction of light propagation and the dipole moment of the absorbing chromophore. This net angular momentum transfer is an additional mechanism that may explain the increase in absorption of R-PE when the ellipticity of the illumination polarization is changed.

Altering the polarization of the illumination to cause increased fluorescence emitted by a fluorophore can have broad applications in flow cytometry and also in other types of fluorescence based instrumentation such as the fluorescence imaging system described further herein. In particular, this technique can increase the measured mean fluorescent intensity (MFI) response of R-PE significantly.

Experimental data is described further herein that was acquired using various lasers. It is to be understood that all of the experimental data included herein does not describe or define a limiting embodiment of the invention. The fluorescence response is measured in units of MFI. A 10-Plex bead set provided by Luminex Corporation was utilized for the experiments. This bead set contains both beads dyed with calibration dye, R-PE attached beads, and a blank bead with no dye. Each bead is identified with a unique region number (e.g., 9-R-PE) that is not related to the MFI expected for that bead. The 10-Plex was measured ten times for each laser using a flow cytometer without alteration of the polarization of the light generated by the lasers. This data set formed the baseline data for both the calibration dye and R-PE.

A waveplate providing π/2 relative retardation (a quarter-waveplate) at 532 nm was then placed between the laser and the laser-focusing lens. Based on prior experiments, the angle of the fast axis of the waveplate was set to approximately 40 degrees with respect to vertical. This configuration changes the linearly polarized illumination to elliptically polarized illumination. Without performing any additional optical alignment, the 10-plex was re-measured by the flow cytometer, and data was collected. In this configuration, the MFI response of R-PE increased, and the MFI response of the calibration dye decreased. The instrument was then recalibrated, and the 10-plex was re-measured ten times. This data provides the amount of MFI increase in the 10 R-PE response with respect to the calibration dye response. The data shown in Table 1 illustrates this response.

In Table 1, No Polarization Optic indicates data measured as described above without alteration of the polarization of the illumination. The first set of Quarter-Wave Plate@40Deg data is data measured using the waveplate described above to alter the polarization of the illumination. The second set of Quarter-Wave Plate@40Deg data is data measured using the waveplate described above to alter the polarization of the illumination after recalibration of the instrument.

TABLE 1

| | 9-R-PE | 17-DYE | 18-R-PE | 45-R-PE | 48-R-PE | 54-DYE | 57-R-PE | 80-Blank | 89-DYE | 90-R-PE |
|---|---|---|---|---|---|---|---|---|---|---|
| No Polarization Optic | 3464 | 390 | 1214 | 736 | 431 | 1031.5 | 85 | 0 | 10336.5 | 5917 |
| No Polarization Optic | 3736 | 386 | 1232 | 736 | 432 | 1015 | 106 | 0 | 10284 | 6004 |
| No Polarization Optic | 3670 | 380.5 | 1332 | 785 | 489.5 | 997 | 89 | 0 | 9857 | 6160.5 |
| No Polarization Optic | 3597 | 375 | 1255 | 741 | 442 | 972.5 | 98 | 0 | 9748 | 6011 |
| No Polarization Optic | 3646 | 369 | 1230 | 743 | 466.5 | 959 | 90 | 0 | 9541 | 5944 |
| Avg No Polarization Optic | 3622.6 | 380.1 | 1252.6 | 748.2 | 452.2 | 995 | 93.6 | 0 | 9953.3 | 6007.3 |
| Quarter-Wave Plate | 3818 | 328.5 | 1337 | 826.5 | 519 | 866.5 | 99 | 0 | 8596 | 6755 |
| @40 Deg | | | | | | | | | | |
| Quarter-Wave Plate | 3930 | 326 | 1437 | 857.5 | 493 | 854 | 106.5 | 0 | 8635 | 6723.5 |
| @40 Deg | | | | | | | | | | |
| Quarter-Wave Plate | 3593 | 320.5 | 1373.5 | 804 | 485.5 | 837 | 98 | 0 | 8637 | 6599 |
| @40 Deg | | | | | | | | | | |
| Avg Quarter-Wave Plate | 3780.3 | 325.0 | 1382.5 | 829.3 | 499.2 | 852.5 | 101.2 | 0.0 | 8622.7 | 6692.5 |
| @40 Deg | | | | | | | | | | |
| % Change | 4.4 | −14.5 | 10.4 | 10.8 | 10.4 | −14.3 | 8.1 | | −13.4 | 11.4 |
| Avg % Change R-PE | 9.2 | | | | | | | | | |
| Avg % Change DYE | −14.1 | | | | | | | | | |
| Recalibration w Quarter- | | | | | | | | | | |
| Wave Plate | | | | | | | | | | |
| Quarter-Wave Plate | 4535 | 372 | 1535.5 | 954 | 575 | 956 | 111.5 | 0 | 9644.5 | 7770.5 |
| @40 Deg | | | | | | | | | | |
| Quarter-Wave Plate | 4414 | 373 | 1632.5 | 939 | 595 | 966 | 120 | 0 | 9683.5 | 7487 |
| @40 Deg | | | | | | | | | | |
| Quarter-Wave Plate | 4437 | 383.5 | 1595 | 968 | 581.5 | 969 | 124 | 0 | 9732 | 7688 |
| @40 Deg | | | | | | | | | | |
| Quarter-Wave Plate | 4655 | 397 | 1610 | 1028 | 591 | 999 | 134 | 0 | 10104 | 8472 |
| @40 Deg | | | | | | | | | | |
| Avg Quarter-Wave Plate | 4510.3 | 381.4 | 1593.3 | 972.3 | 585.6 | 972.5 | 122.4 | | 9791.0 | 7854.4 |
| I@40 Deg | | | | | | | | | | |
| % Change | 24.5 | 0.3 | 27.2 | 29.9 | 29.5 | −2.3 | 30.7 | | −1.6 | 30.7 |
| Avg % Change R-PE | 28.8 | | | | | | | | | |
| Avg % Change DYE | −1.2 | | | | | | | | | |

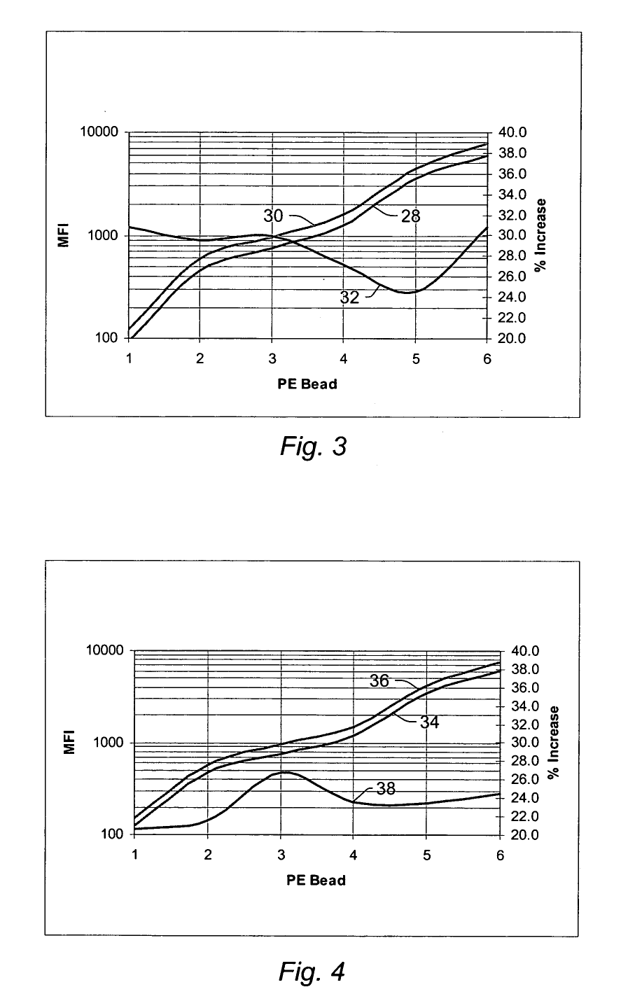

The plots shown in FIGS. 3-6 illustrate the average MFI measured for the six R-PE beads of the bead set described above without the waveplate described above placed in the illumination path and the average MFI measured for the six R-PE beads after the waveplate was placed in the illumination path and the system was recalibrated. The plots shown in FIGS. 3-6 were generated using different lasers on different instruments. The six R-PE beads have varying amounts of R-PE attached to them.

The data shown in FIG. 3 was acquired using a Model No. 4602-010-0485 laser that is commercially available from JDS Uniphase Corporation, San Jose, Calif. Plot 28 illustrates the average MFI measured for each bead without the waveplate placed in the illumination path. Plot 30 illustrates the average MFI measured for each bead while the waveplate is positioned in the illumination path. As shown in FIG. 3, the average MFI measured for each bead without the waveplate placed in the illumination path is lower than the average MFI measured for each bead while the waveplate is positioned in the illumination path. Therefore, the average MFI measured for each of the beads is higher when the waveplate is used to alter the polarization of the light illuminating the beads during the measurements. In addition, as shown in plot 32 of FIG. 3, the % increase in the average MFI measured for each of the beads varies somewhat from bead to bead, but the % increase in the average MFI measured for each of the beads was substantial (e.g., 24.0% or greater). Therefore, the plots shown in FIG. 3 clearly illustrate that the beads exhibit a polarization-induced response in fluorescence emission by R-PE.

The data shown in FIG. 4 was acquired using a Model No. 4602-010-0485 laser that is commercially available from JDS Uniphase Corporation. Plot 34 illustrates the average MFI measured for each bead without the waveplate placed in the illumination path. Plot 36 illustrates the average MFI measured for each bead while the waveplate is positioned in the illumination path. As shown in FIG. 4, the average MFI measured for each bead without the waveplate placed in the illumination path is lower than the average MFI measured for each bead while the waveplate is positioned in the illumination path. Therefore, the average MFI measured for each of the beads is higher when the waveplate is used to alter the polarization of the light illuminating the beads during the measurements. In addition, as shown in plot 38 of FIG. 4, the % increase in the average MFI measured for each of the beads varies somewhat from bead to bead, but the % increase in the average MFI measured for each of the beads was substantial (e.g., 20.0% or greater). Therefore, the plots shown in FIG. 4 clearly illustrate that the beads exhibit a polarization-induced response in fluorescence emission by R-PE.

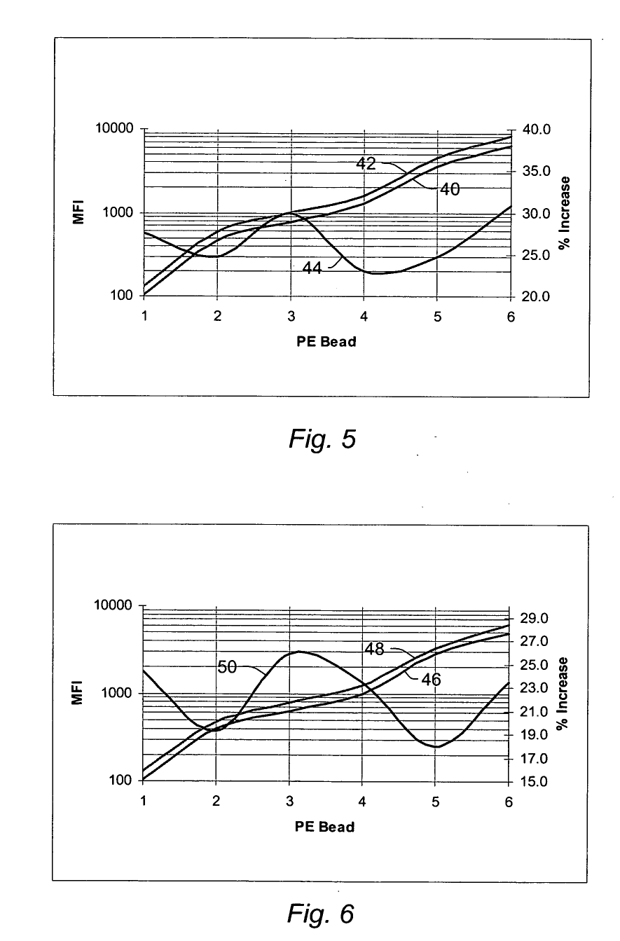

The data shown in FIG. 5 was acquired using a Model No. 4602-010-0485 laser that is commercially available from JDS Uniphase Corporation. Plot 40 illustrates the average MFI measured for each bead without the waveplate placed in the illumination path. Plot 42 illustrates the average MFI measured for each bead while the waveplate is positioned in the illumination path. As shown in FIG. 5, the average MFI measured for each bead without the waveplate placed in the illumination path is lower than the average MFI measured for each bead while the waveplate is positioned in the illumination path. Therefore, the average MFI measured for each of the beads is higher when the waveplate is used to alter the polarization of the light illuminating the beads during the measurements. In addition, as shown in plot 44 of FIG. 5, the % increase in the average MFI measured for each of the beads varies somewhat from bead to bead, but the % increase in the average MFI measured for each of the beads was substantial (e.g., about 22.0% or greater). Therefore, the plots shown in FIG. 5 clearly illustrate that the beads exhibit a polarization-induced response in fluorescence emission by R-PE.

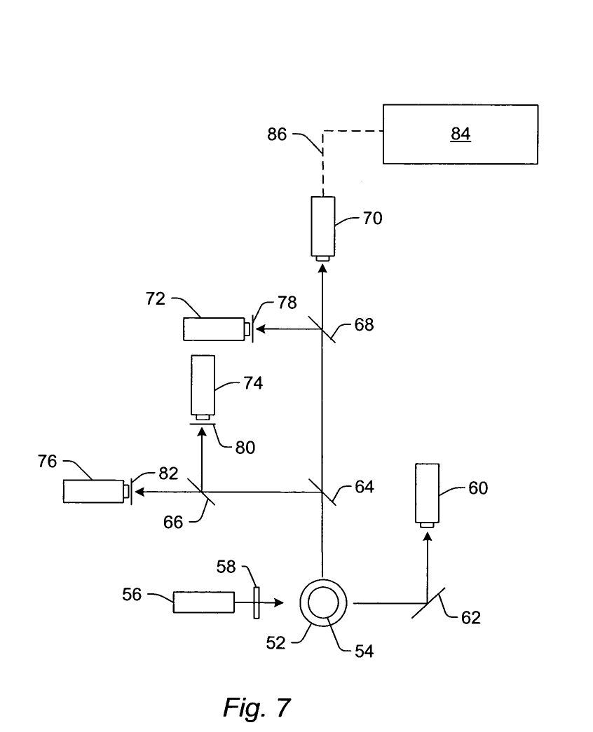

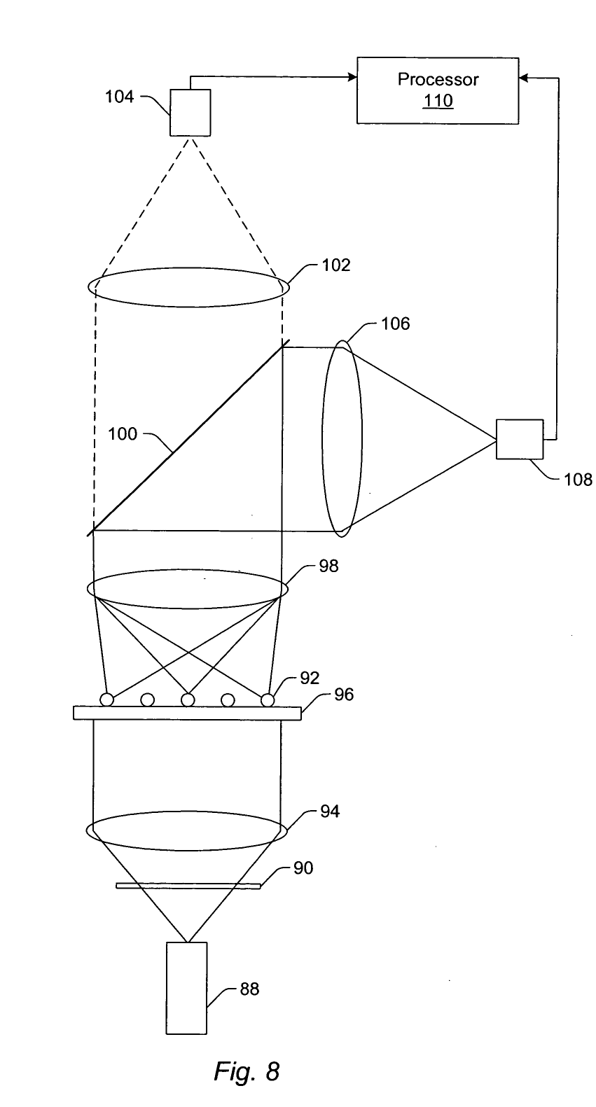

The data shown in FIG. 6 was acquired using a COMPASS 215M-15 laser that is commercially available from Coherent Inc., Santa Clara, Calif. Plot 46 illustrates the average MFI measured for each bead without the waveplate placed in the illumination path. Plot 48 illustrates the average MFI measured for each bead while the waveplate is positioned in the illumination path. As shown in FIG. 6, the average MFI measured for each bead without the waveplate placed in the illumination path is lower than the average MFI measured for each bead while the waveplate is positioned in the illumination path. Therefore, the average MFI measured for each of the beads is higher when the waveplate is used to alter the polarization of the light illuminating the beads during the measurements. In addition, as shown in plot 50 of FIG. 6, the % increase in the average MFI measured for each of the beads varies somewhat from bead to bead, but the % increase in the average MFI measured for each of the beads was substantial (e.g., 17.0% or greater). Therefore, the plots shown in FIG. 6 clearly illustrate that the beads exhibit a polarization-induced response in fluorescence emission by R-PE.

The methods described herein can be implemented in the existing LX100 flow cytometer instrument without major modifications (e.g., by insertion of one or more polarization components into the illumination path) and can easily be implemented on any flow cytometer. The illumination subsystems and methods described herein can increase the amount of fluorescence emitted by fluorophores and thus increase the sensitivity and accuracy of the flow cytometers. This technique can also easily be incorporated into the present flow cytometer instruments without affecting the remaining optical components.

FIG. 7 illustrates one embodiment of a system configured to measure fluorescence of particles. The measurement system illustrated in FIG. 7 includes an illumination subsystem configured according to the embodiments described herein. The embodiment of the system shown in FIG. 7 is configured as a flow cytometer. In FIG. 7, the measurement system is shown along a plane through the cross-section of cuvette 52 through which particles 54 flow. In one example, the cuvette may be a standard fused-silica cuvette such as that used in standard flow cytometers. Any other suitable type of viewing or delivery chamber, however, may also be used to deliver the sample for analysis.

The system includes an illumination subsystem configured to illuminate particles 54 with light having linear polarization oriented in a non-vertical direction, circular polarization, or elliptical polarization. A fluorophore (not shown) is attached to or incorporated into particles 54. The polarization of the light causes fluorescence emitted by the fluorophore to be brighter than fluorescence emitted by the fluorophore when illuminated with linearly polarized light oriented in a predominantly vertical direction or non-polarized light.

The illumination subsystem includes light source 56. In one embodiment, light source 56 is a laser. The laser may be any suitable laser known in the art. In addition, the illumination subsystem may include more than one laser (not shown). The light source may be configured to emit light having one or more wavelengths such as blue light or green light. In other embodiments, the illumination subsystem includes one or more non-laser light sources (not shown) selected from the group consisting of LEDs, arc lamps, fiber illuminators, and light bulbs. The non-laser light source(s) may include any suitable non-laser light source(s) known in the art. In this manner, the illumination subsystem may include more than one light source. In one embodiment, the light sources may be configured to illuminate the particles with light having different wavelengths or wavelength bands (e.g., blue light and green light). In some embodiments, the light sources may be configured to illuminate the particles at different directions. In addition, the illumination subsystem may include one or more lasers and/or one or more non-laser light sources. Light source 56 may include any other appropriate light source known in the art.

The illumination subsystem also includes polarization component 58 disposed in the optical path of the light generated by light source 56. Polarization component 58 may include a half-wave retarder, a quarter-wave retarder, a retarder stack, any other suitable polarization component described herein, or any combination thereof. Polarization component 58 is configured to alter the polarization of the light before the light illuminates particle 54 during measurements performed by the measurement system. For exanple, the light source may be configured to generate linearly polarized light oriented in a predominantly vertical direction or non-polarized light. The altered polarization is linear polarization oriented in a non-vertical direction, circular polarization, or elliptical polarization. In one embodiment, the altered polarization has a polarization ratio of less than 100:1 with any orientation. In another embodiment, the altered polarization has a polarization ratio of greater than 100:1 with a non-vertical orientation. In addition, although the system shown in FIG. 7 includes one polarization component disposed in the optical path between light source 56 and cuvette 52, the system may include more than one polarization component (not shown) disposed in the optical path between light source 56 and cuvette 52. The polarization components may alter the polarization of the light generated by light source 56 in series such that particles 54 are illuminated with light of a selected polarization state and/or orientation.

Light exiting polarization component 58 illuminates the particles as they flow through the cuvette. The illumination causes the particles or a fluorophore attached thereto or incorporated therein to emit fluorescent light having one or more wavelengths or wavelength bands. In addition, the altered polarization of the light exiting the polarization component causes fluorescence emitted by the fluorophore to be brighter than fluorescence emitted by the fluorophore when illuminated with linearly polarized light oriented in a predominantly vertical direction or non-polarized light. In one embodiment, the fluorophore includes R-PE. In a different embodiment, the fluorophore includes an organic or non-organic dye. The organic or non-organic dye may include any appropriate dye known in the art.

In some embodiments, particles 54 themselves are configured to emit fluorescence. In one such embodiment, the polarization of the light exiting polarization component 58 causes the fluorescence emitted by the particles to be brighter than the fluorescence emitted by the particles when illuminated with the linearly polarized light oriented in the predominantly vertical direction or non-polarized light.

In some embodiments, the system may include one or more lenses (not shown) configured to focus light from the light source onto the particles or the flowpath. The one or more lenses may be positioned in the optical path between light source 56 and polarization component 58 or between polarization component 58 and cuvette 52.

Light scattered forwardly from the particles may be directed to detection system 60 by folding mirror 62 or another suitable light directing component. Alternatively, detection system 60 may be placed directly in the path of the forwardly scattered light. In this manner, the folding mirror or other light directing components may not be included in the system. In one embodiment, the forwardly scattered light may be light scattered by the particles at an angle of about 180° from the direction of illumination by light source 56, as shown in FIG. 7. The angle of the forwardly scattered light may not be exactly 180° from the direction of illumination such that incident light from the light source may not impinge upon the photosensitive surface of the detection system. For example, the forwardly scattered light may be light scattered by the particles at angles less than or greater than 180° from the direction of illumination (e.g., light scattered at an angle of about 170°, about 175°, about 185°, or about 190°).

Light scattered by the particles at an angle of about 90° from the direction of illumination may also be collected. Light scattered by the particles can also or alternatively be collected at any angle or orientation. In one embodiment, this scattered light may be separated into more than one beam of light by one or more beamsplitters or dichroic mirrors. For example, light scattered at an angle of about 90° to the direction of illumination may be separated into two different beams of light by beamsplitter 64. The two different beams of light may be separated again by beamsplitters 66 and 68 to produce four different beams of light. Each of the beams of light may be directed to a different detection system, which may include one or more detectors. For example, one of the four beams of light may be directed to detection system 70. Detection system 70 may be configured to detect light scattered by the particles.

Scattered light detected by detection system 60 and/or detection system 70 may generally be proportional to the volume of the particles that are illuminated by the light source. Therefore, output signals of detection system 60 and/or output signals of detection system 70 may be used to determine a diameter of the particles that are in the illumination zone or detection window. In addition, the output signals of detection system 60 and/or detection system 70 may be used to identify more than one particle that are stuck together or that are passing through the illumination zone at approximately the same time. Therefore, such particles may be distinguished from other sample particles and calibration particles.

The system also includes a detection subsystem configured to generate output signals responsive to the fluorescence emitted by the fluorophore. For example, the other three beams of light may be directed to detection systems 72, 74, and 76. Detection systems 72, 74, and 76 may be configured to detect fluorescence emitted by the fluorophore or the particles themselves. Each of the detection systems may be configured to detect fluorescence of a different wavelength or a different range of wavelengths. For example, one of the detection systems may be configured to detect green fluorescence. Another of the detection systems may be configured to detect yellow-orange fluorescence, and the other detection system may be configured to detect red fluorescence.

In some embodiments, spectral filters 78, 80, and 82 may be coupled to detection systems 72, 74, and 76, respectively. The spectral filters may be configured to block fluorescence of wavelengths other than that which the detection systems are configured to detect. In addition, one or more lenses (not shown) may be optically coupled to each of the detection systems. The lenses may be configured to focus the scattered light or emitted fluorescence onto a photosensitive surface of the detectors.

The detector's output current is proportional to the fluorescent light impinging on it and results in a current pulse. The current pulse may be converted to a voltage pulse, low pass filtered, and then digitized by an A/D converter (not shown). Processor 84 such as a digital signal processor (DSP) integrates the area under the pulse to provide a number which represents the magnitude of the fluorescence. As shown in FIG. 7, processor 84 may be coupled to detector 70 via transmission medium 86. Processor 84 may also be coupled to detector 70 indirectly via transmission medium 86 and one or more other components (not shown) such as the A/D converter. The processor may be coupled to other detectors of the system in a similar manner. Processor 84 may be further configured as described herein.

In some embodiments, the output signals generated from fluorescence emitted by the fluorophore or particles may be used to determine an identity of the particles and information about a reaction taken or taking place on the surface of the particles. For example, output signals of two of the detection systems may be used to determine an identity of the particles, and output signals of the other detection system may be used to determine a reaction taken or taking place on the surface of the particles. Therefore, the selection of the detectors and the spectral filters may vary depending on the type of dyes or fluorophores incorporated into or bound to the particles and/or the reaction being measured (i.e., the dye(s) incorporated into or bound to the reactants involved in the reaction).

The detection systems that are used to determine an identity of the sample particles (e.g., detection systems 72 and 74) may be APDs, a PMT, or another type of photodetector. The detection system that is used to identify a reaction taken or taking place on the surface of the particles (e.g., detection system 76) may be a PMT, an APD, or another type of photodetector. The measurement system may be further configured as described herein.

Although the system of FIG. 7 is shown to include two detection systems having two different detection windows for distinguishing between particles having different dye characteristics, it is to be understood that the system may include more than two such detection windows (i.e., 3 detection windows, 4 detection windows, etc.). In such embodiments, the system may include additional beamsplitters and additional detection systems having other detection windows. In addition, spectral filters and/or lenses may be coupled to each of the additional detection systems.

In another embodiment, the system may include two or more detection systems configured to distinguish between different materials that are reacted on the surface of the particles. The different reactant materials may have dye characteristics that are different than the dye characteristics of the particles.

Additional examples of measurement systems that may be configured as described herein (e.g., by insertion of one or more polarization components into the illumination path of the systems) are illustrated in U.S. Pat. Nos. 5,981,180 to Chandler et al., U.S. Pat. No. 6,046,807 to Chandler, U.S. Pat. No. 6,139,800 to Chandler, U.S. Pat. No. 6,366,354 to Chandler, U.S. Pat. No. 6,411,904 to Chandler, U.S. Pat. No. 6,449,562 to Chandler et al., and U.S. Pat. No. 6,524,793 to Chandler et al., which are incorporated by reference as if fully set forth herein. The measurement system described herein may also be further configured as described in these patents. The system shown in FIG. 7 may be further configured as described herein with respect to other systems and embodiments. In addition, the system shown in FIG. 7 has all of the advantages of other embodiments described herein.

Another embodiment of a system configured to measure fluorescence of particles is shown in FIG. 8. The system shown in FIG. 8 may be used in applications such as multi-analyte measurement of a sample. This embodiment of the system is configured as a fluorescence imaging system. The system includes an illumination subsystem configured to illuminate the particles with light having linear polarization oriented in a non-vertical direction, circular polarization, or elliptical polarization. A fluorophore is attached to or incorporated into the particles. The polarization of the light causes fluorescence emitted by the fluorophore to be brighter than fluorescence emitted by the fluorophore when illuminated with linearly polarized light oriented in a predominantly vertical direction or non-polarized light.

In one embodiment, the illumination subsystem includes light source 88. Light source 88 may include one or more light sources such as any suitable LEDs, lasers, arc lamps, fiber illuminators, light bulbs, incandescent lamps, or any other suitable light sources known in the art. In addition, the illumination subsystem may include more than one light source (not shown), each of which is configured to generate light of at least one wavelength or at least one wavelength band. One example of an appropriate combination of light sources for use in the system shown in FIG. 8 includes, but is not limited to, two or more LEDs.

Light generated by more than one light source may be combined into a common illumination path by a beamsplitter (not shown) or any other suitable optical element known in the art such that light from the light sources may be directed to the particles simultaneously. Alternatively, the imaging subsystem may include an optical element (not shown) such as a reflecting mirror and a device (not shown) configured to move the optical element into and out of the illumination path depending on which light source is used to illuminate the particles. In this manner, the light sources may be used to sequentially illuminate the particles with different wavelengths or wavelength bands of light. The light source(s) may also illuminate the substrate from above (not shown), rather than from below the substrate.

The light source(s) may be selected to provide light at wavelength(s) or wavelength band(s) that will cause the particles or materials coupled thereto or incorporated therein to emit fluorescence. For instance, the wavelength(s) or wavelength band(s) may be selected to excite fluorophores, fluorescent dyes, or other fluorescent materials incorporated into the particles and/or coupled to a surface of the particles. In this manner, the wavelength(s) or wavelength band(s) may be selected such that the particles emit fluorescence that is used for classification of the particles. In addition, the wavelength(s) or wavelength band(s) may be selected to excite fluorophores, fluorescent dyes, or other fluorescent materials coupled to the particles via a reagent on the surface of the particles. As such, the wavelength(s) or wavelength band(s) may be selected such that the particles emit fluorescence that is used to detect and/or quantify reaction(s) that have taken place on the surface of the particles.

The illumination subsystem also includes polarization component 90 disposed in an optical path of the light generated by light source 88. Polarization component 90 may include a half-wave retarder, a quarter-wave retarder, a retarder stack, some combination thereof, or any other polarization component or combination of polarization components described herein. Polarization component 90 is configured to alter the polarization of the light before the light illuminates particles 92 during measurements performed by the system. The altered polarization is linear polarization oriented in a non-vertical direction, circular polarization, or elliptical polarization. In one embodiment, the altered polarization has a polarization ratio of less than 100:1 with any orientation. In another embodiment, the altered polarization has a polarization ratio of greater than 100:1 with a non-vertical orientation.

The altered polarization preferably causes fluorescence emitted by the fluorophore to be brighter than fluorescence emitted by the fluorophore when illuminated with linearly polarized light oriented in a predominantly vertical direction or non-polarized light. In one embodiment, the fluorophore includes R-PE. In a different embodiment, the fluorophore includes an organic or non-organic dye. The organic or non-organic dye may include any appropriate dye known in the art. In some embodiments, the particles themselves are configured to emit fluorescence. In one such embodiment, the polarization of the light causes the fluorescence emitted by the particles to be brighter than the fluorescence emitted by the particles when illuminated with the linearly polarized light oriented in the predominantly vertical direction or non-polarized light.

As shown in FIG. 8, the illumination subsystem may include optical element 94 that is configured to direct light from polarization component 90 to substrate 96 on which particles 92 are immobilized. In one example, optical element 94 may be a collimating lens. However, optical element 94 may include any other appropriate optical element that can be used to image light from polarization component 90 onto substrate 96. In addition, although the optical element is shown in FIG. 8 as a single optical element, it is to be understood that optical element 94 may include more than one refractive element. Furthermore, although optical element 94 is shown in FIG. 8 as a refractive optical element, it is to be understood that one or more reflective and/or diffractive optical elements may be used (possibly in combination with one or more refractive optical elements) to image light from polarization component 90 onto substrate 96. In addition, although optical element 94 is shown in FIG. 8 to image light from polarization component 90 onto substrate 96 at a substantially normal angle of incidence, it is to be understood that the system may be configured to direct light to substrate 90 at an oblique angle of incidence.

Furthermore, although polarization component 90 is shown positioned in the optical path between light source 88 and optical component 94, polarization component 90 may alternatively be positioned in the optical path between optical component 94 and substrate 96. In addition, although the system shown in FIG. 8 includes one polarization component disposed in the optical path between light source 88 and substrate 96, the system may include more than one polarization component disposed in the optical path between light source 88 and substrate 96. The polarization components may alter the polarization of the light generated by light source 88 in series such that particles 92 are illuminated with light of a selected polarization state and/or orientation.

Particles 92 may include any of the particles described above. Substrate 96 may include any appropriate substrate known in the art. The particles immobilized on substrate 96 may be disposed in an imaging chamber (not shown) or any other device for maintaining a position of substrate 96 and particles 92 immobilized thereon with respect to the illumination subsystem. The device for maintaining a position of substrate 96 may also be configured to alter a position of the substrate (e.g., to focus the illumination onto the substrate) prior to imaging.

Immobilization of the particles on the substrate may be performed using magnetic attraction, a vacuum filter plate, or any other appropriate method known in the art. Examples of methods and systems for positioning microspheres for imaging are illustrated in U.S. Patent Application Ser. No. 60/627,304 to Pempsell filed Nov. 11, 2004, which is incorporated by reference as if fully set forth herein. The particle immobilization method itself is not particularly important to the method and systems described herein. However, the particles are preferably immobilized such that the particles do no move perceptibly during the detector integration period, which may be multiple seconds long.

The system shown in FIG. 8 also includes a detection subsystem that is configured to generate output signals responsive to the fluorescence emitted by the fluorophore. For example, as shown in FIG. 8, the detection subsystem may include optical element 98 and dichroic beamsplitter 100. Optical element 98 is configured to collect and collimate light from substrate 96 and particles 92 immobilized thereon and to direct the light to beamsplitter 100. Optical element 98 may be further configured as described above with respect to optical element 94. Beamsplitter 100 may include any appropriate beamsplitter known in the art. Beamsplitter 100 may be configured to direct light from optical element 98 to different detectors based on the wavelength of the light. For example, light having a first wavelength or wavelength band may be transmitted by beamsplitter 100, and light having a second wavelength or wavelength band different than the first may be reflected by beamsplitter 100.

The detection subsystem may also include optical element 102 and detector 104. Light transmitted by beamsplitter 100 may be directed to optical element 102. Optical element 102 is configured to focus the light transmitted by the beamsplitter onto detector 104. The detection subsystem may further include optical element 106 and detector 108. Light reflected by beamsplitter 100 may be directed to optical element 106. Optical element 106 is configured to focus the light reflected by the beamsplitter onto detector 108. Optical elements 102 and 106 may be configured as described above with respect to optical element 94.

Detectors 104 and 108 may include, for example, charge coupled device (CCD) detectors or any other suitable imaging detectors known in the art such as CMOS detectors, two-dimensional arrays of photosensitive elements, time delay integration (TDI) detectors, etc. In some embodiments, a detector such as a two-dimensional CCD imaging array may be used to acquire an image of substantially an entire substrate or of all particles immobilized on a substrate simultaneously. The number of detectors included in the system may be equal to the number of wavelengths or wavelength bands of interest such that each detector is used to generate images at one of the wavelengths or wavelength bands.

Each of the images generated by the detectors may be spectrally filtered using an optical bandpass element (not shown) or any other suitable optical element known in the art, which is disposed in the light path from the beamsplitter to the detectors. A different filter “band” may be used for each captured image. The detection wavelength center and width for each wavelength or wavelength band at which an image is acquired may be matched to the fluorescent emission of interest, whether it is used for particle classification or the reporter signal.

In this manner, the detection subsystem of the system shown in FIG. 8 is configured to generate multiple images at different wavelengths or wavelength bands simultaneously. Although the system shown in FIG. 8 includes two detectors, it is to be understood that the system may include more than two detectors (e.g., three detectors, four detectors, etc.). As described above, each of the detectors may be configured to generate images at different wavelengths or wavelength bands simultaneously by including one or more optical elements for directing light at different wavelengths or wavelength bands to the different detectors simultaneously. In addition, although the system is shown in FIG. 8 to include multiple detectors, it is to be understood that the system may include a single detector. The single detector may be used to generate multiple images at multiple wavelengths or wavelength bands sequentially. For example, light of different wavelengths or wavelength bands may be directed to the substrate sequentially, and different images may be generated during illumination of the substrate with each of the different wavelengths or wavelength bands. In another example, different filters for selecting the wavelength or wavelength bands of light directed to the single detector may be altered (e.g., by moving the different filters into and out of the imaging path) to generate images at different wavelengths or wavelength bands sequentially.

The detection subsystem shown in FIG. 8, therefore, is configured to generate a plurality or series of images representing the fluorescent emission of particles 92 at several wavelengths of interest. In addition, the system may be configured to supply a plurality or series of digital images representing the fluorescence emission of the particles to a processor (i.e., a processing engine). In one such example, the system may include processor 110. Processor 110 may be configured to acquire (e.g., receive) image data from detectors 104 and 108. For example, processor 110 may be coupled to detectors 104 and 108 in any suitable manner known in the art (e.g., via transmission media (not shown), each coupling one of the detectors to the processor, via one or more electronic components (not shown) such as analog-to-digital converters, each coupled between one of the detectors and the processor, etc.).

Preferably, processor 110 is configured to process and analyze the images to determine one or more characteristics of particles 92 such as a classification of the particles and information about a reaction taken place on the surface of the particles. The one or more characteristics may be output by the processor in any suitable format such as a data array with an entry for fluorescent magnitude for each particle for each wavelength or wavelength band.

Processor 110 may be a processor such as those commonly included in a typical personal computer, mainframe computer system, workstation, etc. In general, the term “computer system” may be broadly defined to encompass any device having one or more processors, which executes instructions from a memory medium. The processor may be implemented using any other appropriate functional hardware. For example, the processor may include a DSP with a fixed program in firmware, a field programmable gate array (FPGA), or other programmable logic device (PLD) employing sequential logic “written” in a high level programming language such as very high speed integrated circuits (VHSIC) hardware description language (VHDL). In another example, program instructions (not shown) executable on processor 110 may be coded in a high level language such as C#, with sections in C++ as appropriate, ActiveX controls, JavaBeans, Microsoft Foundation Classes (“MFC”), or other technologies or methodologies, as desired. The program instructions may be implemented in any of various ways, including procedure-based techniques, component-based techniques, and/or object-oriented techniques, among others.

The system shown in FIG. 8 may be further configured as described herein with respect to other systems and embodiments. In addition, the system shown in FIG. 8 has all of the advantages of other embodiments described herein.

As described above, the systems shown in FIGS. 7-8 include one or more polarization components disposed in an illumination path of the systems. In addition, as described above, the systems shown in FIGS. 7-8 may include more than one light source. In some such embodiments, some of the light sources may be used to excite fluorescence from different materials coupled to the particles (e.g., a first light source may be used as an excitation source for a classification dye, a second light source may be used as an excitation source for a reporter dye, etc.). In this manner, the polarization of the light generated by each of the light sources may be altered independently to thereby increase the fluorescence of the material that is excited by each of the light sources. For example, a polarization component such as a polarization component described herein may be positioned in the illumination path of the each of the light sources, and if the light beams generated by the light sources are combined into a common illumination path, the polarization components may be disposed in the illumination paths upstream of the optical element(s) that combine(s) the light beams. In this manner, the polarization of each light beam that illuminates the particles may be independently altered and controlled to increase, and possibly even maximize, the magnitude of each (or at least more than one) of the fluorescence measurements performed by the systems.

Further modifications and alternative embodiments of various aspects of the invention may be apparent to those skilled in the art in view of this description. For example, systems, illumination subsystems, and methods for increasing fluorescence emitted by a fluorophore are provided. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A system configured to measure fluorescence of particles, comprising:
an illumination subsystem configured to illuminate the particles with light having linear polarization oriented in a non-vertical direction, circular polarization, or elliptical polarization, wherein a fluorophore is attached to or incorporated into the particles, and wherein the polarization of the light causes fluorescence emitted by the fluorophore to be brighter than fluorescence emitted by the fluorophore when illuminated with linearly polarized light oriented in a predominantly vertical direction or non-polarized light; and
a detection subsystem configured to generate output signals responsive to the fluorescence emitted by the fluorophore.

2. The system of claim 1, wherein the illumination subsystem comprises one or more lasers.

3. The system of claim 1, wherein the illumination subsystem comprises one or more non-laser light sources selected from the group consisting of light emitting diodes, arc lamps, fiber illuminators, and light bulbs.

4. The system of claim 1, wherein the polarization has a polarization ratio of less than 100:1 with any orientation.

5. The system of claim 1, wherein the polarization has a polarization ratio of greater than 100:1 with a non-vertical orientation.

6. The system of claim 1, wherein the fluorophore comprises R-phycoerytherin.

7. The system of claim 1, wherein the fluorophore comprises an organic or non-organic dye.

8. The system of claim 1, wherein the particles are configured to emit fluorescence, and wherein the polarization of the light causes the fluorescence emitted by the particles to be brighter than the fluorescence emitted by the particles when illuminated with the linearly polarized light oriented in the predominantly vertical direction or the non-polarized light.

9. The system of claim 1, wherein the system is further configured as a flow cytometer.

10. The system of claim 1, wherein the system is further configured as a fluorescence imaging system.

11. An illumination subsystem configured to provide illumination for a measurement system, comprising:
a light source configured to generate light; and
a polarization component configured to alter the polarization of the light before the light illuminates particles during measurements performed by the measurement system, wherein the altered polarization is linear polarization oriented in a non-vertical direction, circular polarization, or elliptical polarization, wherein a fluorophore is attached to or incorporated into the particles, and wherein the altered polarization causes fluorescence emitted by the fluorophore to be brighter than fluorescence emitted by the fluorophore when illuminated with linearly polarized light oriented in a predominantly vertical direction or non-polarized light.

12. The illumination subsystem of claim 11, wherein the light source comprises one or more lasers.

13. The illumination subsystem of claim 11, wherein the light source comprises one or more non-laser light sources selected from the group consisting of light emitting diodes, arc lamps, fiber illuminators, and light bulbs.

14. The illumination subsystem of claim 11, wherein the polarization component comprises a half-wave retarder, a quarter-wave retarder, a retarder stack, or some combination thereof.

15. The illumination subsystem of claim 11, wherein the altered polarization has a polarization ratio of less than 100:1 with any orientation.

16. The illumination subsystem of claim 11, wherein the altered polarization has a polarization ratio of greater than 100:1 with a non-vertical orientation.

17. The illumination subsystem of claim 11, wherein the fluorophore comprises R-phycoerytherin.

18. The illumination subsystem of claim 11, wherein the fluorophore comprises an organic or non-organic dye.

19. The illumination subsystem of claim 11, wherein the measurement system is configured as a flow cytometer.

20. The illumination subsystem of claim 11, wherein the measurement system is configured as a fluorescence imaging system.

21. A method for increasing fluorescence emitted by a fluorophore attached to or incorporated into particles, comprising altering the polarization of light before the light illuminates the particles during a measurement, wherein the altered polarization is linear polarization oriented in a non-vertical direction, circular polarization, or elliptical polarization.

22. The method of claim 21, wherein the altered polarization has a polarization ratio of less than 100:1 with any orientation.

23. The method of claim 21, wherein the altered polarization has a polarization ratio of greater than 100:1 with a non-vertical orientation.
